(19) United States
(12) Patent Application Publication
(10) Pub. No.: US 2026/0053730 A1
(43) Pub. Date: Feb. 26, 2026

(54) SEMIFLUORINATED ALKANE TETRACYCLINE COMBINATION COMPOSITIONS FOR TREATMENT OF MALAR EDEMA

(71) Applicant: Bausch + Lomb Ireland Limited, Dublin (IE)

(72) Inventor: Anuradha Gore, Aliso Viejo, CA (US)

(73) Assignee: Bausch + Lomb Ireland Limited, Dublin (IE)

(21) Appl. No.: 19/303,894

(22) Filed: Aug. 19, 2025

Related U.S. Application Data
(60) Provisional application No. 63/685,183, filed on Aug. 20, 2024.

(51) Int. Cl. A61K 9/00; A61K 9/08; A61K 31/02; A61K 31/65; A61K 45/06; A61K 47/06

(52) U.S. Cl. CPC A61K 9/0019; A61K 9/08; A61K 31/02; A61K 31/65; A61K 45/06; A61K 47/06

(57) ABSTRACT

Disclosed herein are injectable compositions, such as nonaqueous liquid injectable compositions, comprising at least one tetracycline antibiotic, such as doxycycline, and at least one semifluorinated alkane, such as 1-perfluorohexyloctane (F6H8), as well as methods of treatment or prevention of malar edema, with such compositions. The injectable compositions may further be provided in a container closure system, such as a polypropylene (PP) pre-filled syringe, or in a kit.

Disclosed herein are injectable compositions, such as nonaqueous liquid injectable compositions, comprising at least one tetracycline antibiotic, such as doxycycline, and at least one semifluorinated alkane, such as 1-perfluorohexyloctane (F6H8). The injectable compositions may be used to treat and/or temporarily prevent malar edema, in a single-dose unit composition. The injectable compositions are stable for each active ingredient, provide efficient drug delivery, and are well tolerated by the eye. The injectable compositions may be provided in a vial, single dose syringe, or in a kit.

Typically, injectable compositions are administered as subcutaneous injections, with one or more injections of the composition being applied to an eye of the subject suffering from or susceptible to malar edema one or more times per year, although the frequency of administration of such compositions may be dependent on multiple factors, including the makeup of the particular composition and the volume of the edema, mound, or festoon to which the compositions are injected.

Of particular importance for efficacy and commercialization of injectable compositions is stability. Maintenance of efficacy and stability of injectable compositions may be required to meet various federal health and safety regulations, e.g., potency, impurities, shelf life testing, sterility, etc. For example, injectable compositions may be required to contain expiration dates posted on their container, which may be predicated on the stability of the active ingredients and other conditions inherent in the formulation and environmental exposures of the product.

Furthermore, it may be desirable for an injectable composition to include a plurality of active agents. In such situations, it may be difficult or uneconomical to meet a particular shelf life target or regulatory requirements due to some instability of one or more of the combination of the active agents or other interaction with components of a formulation. This may be the result of some chemical reactivity or incompatibility of one or more of the active agents, for example, which leads to degradation of one or more of the active agents. Such degradation shortens the shelf life of the composition and may render the formulation pharmaceutically ineffective or non-compliant with federal regulatory requirements.

Certain tetracycline antibiotics, such as doxycycline, are known to be effective against bacteria and/or to have other properties useful in the effective treatment of various conditions, but are not sufficiently stable, for example in an aqueous solution at neutral pH, and are therefore unsuitable for use in, for example, pre-filled syringes. Tetracyclines, like this, are only suitable for use in products that are either reconstituted in an aqueous form just prior to use. There is, thus, a need for improved injectable compositions comprising antibiotic agents such as doxycycline, where the product is ready-to-use without a need for reconstitution and can be conveniently injected. For example, a tetracycline antibiotic suspension in a nonaqueous liquid injectable composition that may be administered as an injection.

The present inventors have surprisingly discovered that improved, stable injectable compositions may be provided comprising (a) at least one tetracycline antibiotic and (b) at least one semifluorinated alkane. Without wishing to be bound by theory, it is believed that, not only does the at least one semifluorinated alkane provide its own malar edema and/or prevention effect, but it also acts as a stable, non-aqueous vehicle or carrier for the at least one tetracycline antibiotic. It is thus the unique combination of the at least one tetracycline antibiotic and the at least one semifluorinated alkane that provides the improved, stable injectable compositions described herein. In some embodiments, the injectable compositions may be nonaqueous liquid compositions comprising (a) at least one tetracycline antibiotic, such as doxycycline, and (b) at least one semifluorinated alkane, such as 1-perfluorohexyloctane (F6H8).

Further disclosed herein are methods for the treatment and/or temporary prevention of malar edema, comprising administering via injection to a patient in need thereof an effective amount of an injectable composition comprising (a) at least one tetracycline antibiotic, such as doxycycline, and (b) at least one semifluorinated alkane, such as 1-perfluorohexyloctane (F6H8).

These and other features, aspects, and advantages of the disclosure will be apparent from a reading of the following.

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. Numeric values modified by the term “about” include the specific identified value. For example, “about 100” means a number ranging from 95 to 105, including 95, 100, and 105. Recitation of ranges of values herein are merely intended to serve as a shorthand method of referring individually to each separate value falling within the range, unless otherwise indicated herein, and each separate value is incorporated into the specification as if it were individually recited herein.

As used herein, “administration” of a compound or composition to a patient refers to any route of introducing or delivering the compound or composition to a subject. Administration includes self-administration and the administration by another.

As used herein, a “condition,” “disorder,” or “disease” relates to any unhealthy or abnormal state.

As used herein, an “effective amount” or “effective dose” refers to an amount of a compound or composition that treats and/or temporarily prevents, upon single or multiple dose administration, a patient suffering from a disorder, disease, or condition. An effective amount can be determined by the attending diagnostician through the use of known techniques and by observing results obtained under analogous circumstances. In determining the effective amount, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, a composition is “free” of a component when said component is not intentionally added during manufacture of the composition.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, the terms “patient,” “subject,” “individual,” and the like, as used herein, are interchangeable and refer to any animal, which may be a human or a non-human animal.

As used herein, the terms “prevention” of or “preventing” a disorder, disease, or condition refers to reduction of or reducing the occurrence of the disorder, disease, or condition in a treated sample relative to an untreated control sample, and includes delaying onset, progression, or reduction of severity of one or more symptoms of the disorder or condition relative to the untreated control sample.

As used herein, the term “malar edema” refers to any of the signs, symptoms, and/or underlying cause(s) of the swelling of the lower third of the orbicularis oculi muscle, such as bulging, discoloration, or sagging around or below the eye, including malar mounds or festoons.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which, in the aggregate, can serve as a vehicle or medium for active ingredients. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, a composition according to the present disclosure is “stable” if the at least one tetracycline antibiotic within the composition degrades by less than about 10% after being stored at about 25° C. for 10 weeks, as measured by high-performance liquid chromatography (HPLC) or an equivalent appropriate technique such as that within the U.S. Pharmacopeia.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As will be understood by one of ordinary skill in the art, each range disclosed herein includes all possible subranges as well as individual numerical values within that range, including endpoints. As a non-limiting example, a range of “0.001% to 0.02%” includes and would be understood to specifically disclose subranges such as “0.004% to 0.01%,” “0.005% to 0.02%,” etc., as well as all individual numbers within the disclosed range, for example, 0.001%, 0.004%, 0.005%, 0.01%, 0.02%, etc.

Claims or descriptions that include “or” or “and/or” between members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

The injectable compositions of the present disclosure comprise (a) at least one tetracycline antibiotic and (b) at least one semifluorinated alkane. The at least one tetracycline antibiotic may be chosen from any tetracycline compound (herein “a tetracycline” or “tetracyclines”) or a pharmaceutically acceptable salt or hydrate thereof substantially stabilized in a base. The tetracyclines are characterized by a carbon skeleton composed of four linearly fused six-membered carbon rings (octahydrotetracene-2-carboxamide skeleton). They are defined as “a subclass of polyketides having an octahydrotetracene-2-carboxamide skeleton”. They are collectively known as “derivatives of polycyclic naphthacene carboxamide.” Unless specifically indicated herein, the term tetracycline, as well as individually named antibiotics, are to be understood to include the free base and any salts, hydrates, solvates, and hyclates thereof. For example, the term doxycycline is to be understood to include the free base and any salts, hydrates, solvates, and hyclates thereof, for example an HCL salt, mono-hydrate, or hyclate (hydrochloride hemiethanolate hemihydrate) thereof.

In some embodiments, the at least one tetracycline antibiotic comprises at least one tetracycline derivative chosen from chlortetracycline, oxytetracycline doxycycline, demeclocycline, minocycline, tetracycline, lymecycline, meclocycline, methacycline, rolitetracycline, tigecycline, or any combination thereof.

In some embodiments, the at least one tetracycline antibiotic comprises doxycycline. In some embodiments, the at least one tetracycline antibiotic consists essentially of doxycycline.

In some embodiments, the at least one tetracycline antibiotic, for example doxycycline, is included in the composition at a concentration from about 0.005% (w/w) to about 5% (w/w), for example, from about 0.02% (w/w) to about 4% (w/w), from about 0.5% (w/w) to about 3% (w/w), or from about 0.75% (w/w) to about 2% (w/w).

The injectable compositions of the present disclosure comprise at least one semifluorinated alkane (SFA). Semifluorinated alkanes are linear or branched alkanes some of whose hydrogen atoms have been replaced by fluorine. In particular, semifluorinated alkanes are compounds composed of at least one perfluorinated hydrocarbon segment and at least one non-fluorinated hydrocarbon segment. According to a nomenclature used herein, the semifluorinated alkanes may be referred to as FnHm, wherein F refers to the perfluorinated hydrocarbon segment (F-segment), H refers to the non-fluorinated hydrocarbon segment (H-segment), and n and m refer to the number of carbon atoms of those segments, respectively. For example, F3H3 is used for perfluoropropylpropane and F4H5 is used for 1-perfluorobutylpentane. This type of nomenclature is usually used for compounds having linear segments. Therefore, unless otherwise indicated, it should be assumed that F3H3 means 1-perfluoropropylpropane, rather than 2-perfluoropropylpropane, 1-perfluoroisopropylpropane, or 2-perfluoroisopropylpropane.

In some embodiments, the at least one semifluorinated alkane may have one non-fluorinated hydrocarbon segment attached to one perfluorinated hydrocarbon segment, according to the general formula FnHm. In some embodiments, the at least one semifluorinated alkane is a compound according to the formula FnHm, wherein the F-segment and the H-segment are linear or branched and wherein n and m, which may be the same or different, are independently chosen from 3 to 20 carbon atoms, for example from 3 to 10 carbon atoms, or from 4 to 8 carbon atoms. In some embodiments, the at least one semifluorinated alkane is a compound according to the formula FnHm, wherein the F-segment and the H-segment are linear and wherein n and m, which may be the same or different, are independently chosen from 3 to 20 carbon atoms, for example from 3 to 10 carbon atoms, or from 4 to 8 carbon atoms.

In some embodiments, the ratio of the carbon atoms of the F-segment to the carbon atoms of the H-segment (said ratio obtained by dividing the number of carbon atoms in the F-segment by the number of carbon atoms in the H-segment) of the at least one linear or branched semifluorinated alkane ranges between 0.5 and 3.0, for example between 0.6 and 1.0. For example, the ratio of the carbon atoms of the F-segment to the carbon atoms of the H-segment for 1-perfluorohexyloctane (F6H8) is 0.75. In some embodiments, the at least one semifluorinated alkane is a linear semifluorinated alkane according to the formula FnHm, wherein the ratio of the carbon atoms of the F-segment to the carbon atoms of the H-segment ranges between 0.5 and 3.0, for example between 0.6 and 1.0.

In some embodiments, the at least one semifluorinated alkane may have two perfluorinated hydrocarbon segments separated by one non-fluorinated hydrocarbon segment, according to the general formula FnHmFo, wherein n, m, and o refer to the number of carbon atoms of the first perfluorinated hydrocarbon segment, the non-fluorinated hydrocarbon segment, and the second perfluorinated hydrocarbon segment, respectively. In some embodiments, the at least one semifluorinated alkane is a compound according to the formula FnHmFo, wherein the F-segments and the H-segment are linear or branched and wherein n, m, and o, which may be the same or different, are independently chosen from 3 to 20 carbon atoms, for example from 3 to 10 carbon atoms, or from 4 to 8 carbon atoms.

In some embodiments, the at least one semifluorinated alkane may be a linear semifluorinated alkane comprising a branched non-fluorinated hydrocarbon segment comprising one or more alkyl groups chosen from —CH₃, —C₂H₅, —C₃H₇, —C₄H₉, or any combination thereof. In some embodiments, the at least one semifluorinated alkane may be a linear semifluorinated alkane comprising a branched perfluorinated hydrocarbon segment comprising one or more perfluorinated alkyl groups chosen from —CF₃, —C₂F₅, —C₃F₇, —C₄F₉, or any combination thereof.

In some embodiments, the at least one semifluorinated alkane may comprise a combination of more than one semifluorinated alkane.

In some embodiments, the at least one semifluorinated alkane is chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof. In some embodiments, the at least one semifluorinated alkane is chosen from F4H4, F4H5, F4H6, F5H5, F5H6, F5H7, F5H8, F6H6, F6H7, F6H8, F6H9, F6H10, F8H8, F8H10, F8H12, F10H10, or any combination thereof. In some embodiments, the at least one semifluorinated alkane is chosen from F4H5, F4H6, F5H6, F5H7, F6H6, F6H7, F6H8, or any combination thereof.

In some embodiments, the at least one semifluorinated alkane comprises 1-perfluorohexyl-octane (F6H8). In some embodiments, the at least one semifluorinated alkane consists essentially of 1-perfluorohexyl-octane (F6H8).

The compositions disclosed herein may be used for the treatment or temporary prevention of malar edema.

Depending on the at least one tetracycline antibiotic chosen for the ophthalmic composition, the at least one semifluorinated alkane may further be chosen from any semifluorinated alkane that provides a vehicle or carrier for the at least one tetracycline antibiotic that results in a stable composition. In view of the description herein, it is within the skill of those in the art to choose a semifluorinated alkane that may result in a stable composition with a chosen antibiotic.

In some embodiments, the injectable composition comprises (a) at least one tetracycline antibiotic chosen from doxycycline, demeclocycline, minocycline, tetracycline, or any combination thereof and (b) at least one semifluorinated alkane chosen from F4H5, F4H6, F5H6, F5H7, F6H6, F6H7, F6H8, or any combination thereof.

In some embodiments, the injectable composition comprises (a) at least one tetracycline antibiotic comprising doxycycline and (b) at least one semifluorinated alkane comprising F6H8.

In some embodiments, the injectable composition comprises (a) at least one tetracycline antibiotic, wherein the at least one tetracycline antibiotic consists essentially of doxycycline, and (b) at least one semifluorinated alkane, wherein the at least one semifluorinated alkane consists essentially of F6H8.

In some embodiments, the injectable composition consists essentially of (a) at least one tetracycline antibiotic, wherein the at least one tetracycline antibiotic consists essentially of doxycycline, and (b) at least one semifluorinated alkane, wherein the at least one semifluorinated alkane consists essentially of F6H8.

In some embodiments, the injectable compositions of the present disclosure are liquid composition. In some embodiments, the injectable compositions of the present disclosure are liquid compositions comprising water. In preferred embodiments, the injectable compositions of the present disclosure are liquid compositions that are substantially water-free (nonaqueous).

In some embodiments, the injectable compositions of the present disclosure are stable liquid compositions. In some embodiments, the injectable compositions of the present disclosure are stable nonaqueous liquid compositions.

In some embodiments, the injectable compositions of the present disclosure may be administered in combination with an effective amount of an additional therapeutic agent. In some embodiments, the injectable compositions of the present disclosure may be administered in combination with an effective amount of at least one additional therapeutic agent chosen from redness reduction agents, NSAIDS, steroids, local anesthetics, antihistamines, anti-inflammatory agents, anti-allergens, or any combination thereof.

In some embodiments, the injectable compositions of the present disclosure further comprise, in the same composition, an effective amount of an additional therapeutic agent. In some embodiments, the injectable compositions of the present disclosure further comprise an effective amount of at least one additional therapeutic agent chosen from redness reduction agents, NSAIDS, steroids, local anesthetic, antihistamines, anti-inflammatory agents, anti-allergens, or any combination thereof.

In some embodiments, the injectable compositions of the present disclosure may further include at least one additional non-therapeutic component and/or pharmaceutically acceptable excipient, including, but not limited to, tonicity agents, preservatives, co-solvents, buffers, pH adjustors, antioxidants, delivery vehicles, stabilizers, suspending agents, viscosity-increasing agents, wetting agents, solubilizing agents, chelating agents, nitrous oxide inhibitors, isotonic agents, humectants, surfactants, and the like. The proportion and nature of any additional non-therapeutic component and/or pharmaceutically acceptable excipient may be determined by the chosen route of administration and standard pharmaceutical practice. One of ordinary skill in the art can readily select the proper form and route of administration depending upon the disorder or condition to be treated, the stage of the disorder or condition, and other relevant circumstances.

In some embodiments, the injectable compositions of the present disclosure are sterile, according to USP/EP criteria. In some embodiments, sterility is conferred by any conventional method. In some embodiments, sterility is conferred by filtration. In some embodiments, sterility is conferred by irradiation. In some embodiments, sterility is conferred by heating. In some embodiments, sterility is conferred by conducting the manufacturing process under aseptic conditions.

Further disclosed herein are methods for the treatment or temporary prevention of malar edema comprising administering via injection to a patient in need thereof an effective amount of an injectable composition as described above comprising (a) at least one tetracycline antibiotic and (b) at least one semifluorinated alkane.

In some embodiments, the methods of the present disclosure comprise treatment or temporary prevention of malar edema comprising administering via injection to a patient in need thereof an effective amount of an injectable composition comprising (a) at least one tetracycline antibiotic chosen from doxycycline, demeclocycline, minocycline, tetracycline, or any combination thereof and (b) at least one semifluorinated alkane chosen from F4H5, F4H6, F5H6, F5H7, F6H6, F6H7, F6H8, or any combination thereof.

In some embodiments, the methods of the present disclosure comprise treatment or temporary prevention of malar edema comprising administering via injection to a patient in need thereof an effective amount of an injectable composition comprising (a) at least one tetracycline antibiotic comprising doxycycline and (b) at least one semifluorinated alkane comprising F6H8.

In some embodiments, the methods of the present disclosure comprise treatment or temporary prevention of malar edema comprising administering via injection to a patient in need thereof an effective amount of an injectable composition comprising (a) at least one tetracycline antibiotic, wherein the at least one tetracycline antibiotic consists essentially of doxycycline, and (b) at least one semifluorinated alkane, wherein the at least one semifluorinated alkane consists essentially of F6H8.

In some embodiments, the injectable compositions can be administered by any convenient route. In some embodiments, the injectable compositions are any liquid form suitable for injection. In some embodiments, the injectable compositions are injected subcutaneously. In some embodiments, the injectable compositions are any liquid form suitable for injection. In some embodiments, the injectable compositions are injected intradermally. In some embodiments, the injectable compositions are any liquid form suitable for injection. In some embodiments, the injectable compositions are injected intra-muscularly. In some embodiments, the composition may be in the form of a liquid (such as a suspension, a solution, an emulsion, or the like) or in the form of an ointment. In some embodiments, the injectable composition may be in the form of a suspension. In some embodiments, the injectable composition may be in the form of a solution.

Proper dosages of the injectable compositions of the present disclosure are concentration-dependent. To determine the specific dose, a skilled artisan would have to take into account kinetics and absorption characteristics of the particular ingredients. In addition, the dosage may be dependent on the route of injection. The dosages may also de dependent on the degree of aesthetic effect desired in a patient.

Methods of treating comprise administration of an annual dose of an injectable composition of the present disclosure to a subject in need thereof. Determining and adjusting an appropriate dosing regimen (e.g., adjusting the number of doses and frequency of dosing) per year can be performed by one of ordinary skill in the relevant art, and will depend upon various factors such as the nature and progression of a disorder and/or condition associated with allergies and eye redness, the health, and/or age of the subject. In some embodiments, the injectable compositions of the present disclosure are administered to a patient in a single dose per year, in two doses per year, in three doses per year, in four doses per year, etc. or up to, for example, twelve doses per day. In some embodiments, the composition is administered to a patient at least once, at least twice, at least three times, at least four times, at least 2 months, at least 3 months, at least 4 months, at least 5 months, at least 6 months, at least 12 months, at least one year, or for more than one year.

Further disclosed herein are container closure systems containing an injectable composition as described above comprising (a) at least one tetracycline antibiotic and (b) at least one semifluorinated alkane.

The injectable compositions disclosed herein may be kept in container closure systems (e.g., vials, ampoules, bottles, tubes, syringes, dispenser packages, or other suitable containers). In some embodiments, the compositions may be packaged either in a single-dose unit container closure system or multi-dose container closure system. In some embodiments, the compositions may be packaged in a single-dose unit container closure system. Container closure systems may be composed of one or more materials appropriate for its use, such as aluminum, glass, polypropylene, polyethylene (e.g., low-density polyethylene and high-density polyethylene), polyethylene terephthalate, and polyethylene terephthalate glycol. Plastic container closure systems weigh less, are more resistant to shock and other mechanical influences, cost less, and offer more design possibilities than glass. Polyethylene, e.g., low-density polyethylene (LDPE), without or with additives, and polypropylene (PP) are the plastics required by the European Pharmacopoeia. In some embodiments, the container closure system comprises polypropylene. In some embodiments, the container closure system is composed of polypropylene.

In some embodiments, a single container closure system can comprise one or more compartments for containing a provided injectable composition, and/or appropriate carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated.

Injectable compositions disclosed herein may be prepared according to any known method for the manufacture of ophthalmic formulations or preparations. As will be appreciated by those of ordinary skill in the art, a number of methods are known. In some embodiments, the injectable compositions disclosed herein may be prepared by any conventional technique, such as, e.g., those described in Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York.

In some embodiments, the injectable compositions of the present disclosure are in polypropylene (PP) container closure systems (e.g., vials, ampoules, bottles, tubes, syringes, dispenser packages, or other suitable containers). Exemplary PP container closure systems include white PP syringes and natural PP syringes.

In some embodiments, the present disclosure provides a container closure system containing an injectable composition comprising (a) at least one tetracycline antibiotic and (b) at least one semifluorinated alkane as disclosed above, wherein the container closure system is a pre-filled syringe.

The present disclosure also provides a kit comprising a formulation container containing an injectable composition comprising (a) at least one tetracycline antibiotic and (b) at least one semifluorinated alkane as disclosed above, wherein the container closure system is a pre-filled syringe. In some embodiments, kits can include a second container (the solvent container) comprising a suitable delivery vehicle for dilution or suspension of the provided injectable composition in the first container (the formulation container) for preparation of administration to a subject. In some embodiments, the contents of the formulation container and contents of the solvent container combine to form at least one unit dosage form. In some embodiments, the solvent container contains a delivery vehicle that is aqueous. In some embodiments, the solvent container contains a delivery vehicle that is nonaqueous. Suitable delivery vehicle for the solvent container are detailed above.

In some embodiments, a single container can comprise one or more compartments for containing a provided ophthalmic composition, and/or appropriate vehicle carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated. A kit can thus comprise such multi-compartment containers including the injectable compositions disclosed herein and an appropriate solvent and/or an appropriate vehicle carrier for suspension.

Without limitation, some embodiments of the disclosure include:

### EXEMPLARY COMPOSITION EMBODIMENTS

Embodiment 1. An injectable composition comprising: (a) at least one tetracycline antibiotic and (b) at least one semifluorinated alkane.

Embodiment 2. The injectable composition according to any one of Embodiments 1-2, wherein the at least one tetracycline antibiotic comprises at least one tetracycline derivative chosen from doxycycline, demeclocycline, minocycline, tetracycline, or any combination thereof.

Embodiment 3. The injectable composition according to any one of Embodiments 1-2, wherein the at least one tetracycline antibiotic comprises doxycycline.

Embodiment 4. The injectable composition according to any one of Embodiments 1-3, wherein the at least one tetracycline antibiotic consists essentially of doxycycline.

Embodiment 5. The injectable composition according to any one of Embodiments 1-4, wherein the at least one tetracycline antibiotic is included in the composition at a concentration from about 0.005% (w/w) to about 5% (w/w), for example, from about 0.02% (w/w) to about 4% (w/w), from about 0.5% (w/w) to about 3% (w/w), or from about 0.75% (w/w) to about 2% (w/w).

Embodiment 6. The injectable composition according to any one of Embodiments 1-5, wherein the at least one semifluorinated alkane is a compound composed of at least one perfluorinated hydrocarbon segment (F-segment) and at least one non-fluorinated hydrocarbon segment (H-segment).

Embodiment 7. The injectable composition according to any one of Embodiments 1-6, wherein the at least one semifluorinated alkane is a compound composed of F-segments and H-segments according to the formula FnHm, wherein the F-segments and the H-segments are linear or branched and wherein n and m, which may be the same or different, are independently chosen from 3 to 20 carbon atoms, or from 3 to 10 carbon atoms, or from 4 to 8 carbon atoms.

Embodiment 8. The injectable composition according to any one of Embodiments 1-6, wherein the at least one semifluorinated alkane is a compound composed of F-segments and H-segments according to the formula FnHm, wherein the F-segments and the H-segments are linear and wherein n and m, which may be the same or different, are independently chosen from 3 to 20 carbon atoms, or from 3 to 10 carbon atoms, or from 4 to 8 carbon atoms.

Embodiment 9. The injectable composition according to any one of Embodiments 1-6, wherein the at least one semifluorinated alkane is a compound composed of F-segments and H-segments according to the formula FnHmFo, wherein the F-segments and H-segments are linear or branched and wherein n, m, and o, which may be the same or different, are independently chosen from 3 to 20 carbon atoms, or from 3 to 10 carbon atoms, or from 4 to 8 carbon atoms.

Embodiment 10. The injectable composition according to any one of Embodiments 1-6, wherein the ratio of the carbon atoms of the F-segment to the carbon atoms of the H-segment of the at least one linear or branched semifluorinated alkane ranges between 0.6 and 3.0 or between 0.6 and 1.0.

Embodiment 11. The injectable composition according to any one of Embodiments 1-6, wherein the ratio of the carbon atoms of the F-segment to the carbon atoms of the H-segment of the at least one linear semifluorinated alkane ranges between 0.6 and 3.0 or between 0.6 and 1.0.

Embodiment 12. The injectable composition according to any one of Embodiments 1-6, wherein the at least one semifluorinated alkane is chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof.

Embodiment 13. The injectable composition according to any one of Embodiments 1-6, wherein the at least one semifluorinated alkane is chosen from F4H4, F4H5, F4H6, F5H5, F5H6, F5H7, F5H8, F6H6, F6H7, F6H8, F6H9, F6H10, F8H8, F8H10, F8H12, F10H10, or any combination thereof.

Embodiment 14. The injectable composition according to any one of Embodiments 1-6, wherein the at least one semifluorinated alkane is chosen from F4H5, F4H6, F5H6, F5H7, F6H6, F6H7, F6H8, or any combination thereof.

Embodiment 15. The injectable composition according to any one of Embodiments 1-6, wherein the at least one semifluorinated alkane comprises 1-perfluorohexyl-octane (F6H8).

Embodiment 16. The injectable composition according to any one of Embodiments 1-12, wherein the at least one semifluorinated alkane consists essentially of 1-perfluorohexyl-octane (F6H8).

Embodiment 17. The injectable composition according to any one of Embodiments 1-16, wherein (a) the at least one tetracycline antibiotic is chosen from doxycycline, demeclocycline, minocycline, tetracycline, or any combination thereof and (b) the at least one semifluorinated alkane is chosen from F4H5, F4H6, F5H6, F5H7, F6H6, F6H7, F6H8, or any combination thereof.

Embodiment 18. The injectable composition according to any one of Embodiments 1-17, wherein (a) the at least one tetracycline antibiotic comprises doxycycline and (b) the at least one semifluorinated alkane comprises F6H8.

Embodiment 19. The injectable composition according to any one of Embodiments 1-18, wherein (a) the at least one tetracycline antibiotic consists essentially of doxycycline and (b) the at least one semifluorinated alkane consists essentially of F6H8.

Embodiment 20. The injectable composition according to any one of Embodiments 1-19, wherein the composition is liquid.

Embodiment 21. The injectable composition according to any one of Embodiments 1-20, wherein the composition is nonaqueous.

Embodiment 22. The injectable composition according to any one of Embodiments 1-21, wherein the composition is in the form of a suspension, a solution, or an emulsion.

Embodiment 23. The injectable composition according to any one of Embodiments 1-22, wherein the composition is stable.

Embodiment 24. The injectable composition according to any one of Embodiments 1-23, wherein the composition further comprises at least one additional therapeutic agent.

Embodiment 25. The injectable composition according to any one of Embodiments 1-24, wherein the composition further comprises at least one non-therapeutic component and/or pharmaceutically acceptable excipient.

Embodiment 26. The injectable composition according to any one of Embodiments 1-25, wherein the composition further comprises at least one additional therapeutic agent chosen from redness reduction agents, NSAIDS, steroids, local anesthetic, antihistamines, anti-inflammatory agents, anti-allergens, or any combination thereof.

Embodiment 27. The injectable composition according to any one of Embodiments 1-26, wherein the at least one additional therapeutic agent is a local anesthetic.

Embodiment 28. The injectable composition according to any one of Embodiments 1-27, wherein the at local anesthetic is lidocaine.

Embodiment 29. The injectable composition according to any one of Embodiments 1-28, wherein the composition further comprises at least one non-therapeutic component and/or pharmaceutically acceptable excipient chosen from tonicity agents, co-solvents, preservatives, buffers, pH adjustors, antioxidants, delivery vehicles, stabilizers, suspending agents, viscosity-increasing agents, wetting agents, solubilizing agents, chelating agents, nitrous oxide inhibitors, isotonic agents, humectants, surfactants, or any combination thereof.

### Exemplary Method of Treatment Embodiments:

Embodiment 30. A method for the treatment or temporary prevention of malar edema comprising administering via injection to a patient in need thereof an effective amount of an injectable composition comprising (a) at least one tetracycline antibiotic and (b) at least one semifluorinated alkane.

Embodiment 31. The method according to Embodiment 30, wherein the injectable composition is the injectable composition according to any of Embodiments 1-29.

Embodiment 32. The method according to any one of Embodiments 30-31, wherein the injectable composition is injected into the area of the malar edema of the patient.

Embodiment 33. The method according to any one of Embodiments 30-32, wherein the injectable composition is injected in the form of a suspension, an emulsion, or a solution.

Embodiment 34. The method according to any one of Embodiments 30-33, wherein the injectable composition is administered to the patient in a single dose per year, in two doses per year, in three doses per year, in four doses per year, or in up to twelve doses per year.

Embodiment 35. The method according to any one of Embodiments 30-34, wherein the injectable composition is administered to the patient at least once, at least twice, at least three times, at least one month, at least 2 months, at least 3 months, at least 4 months, at least 5 months, at least 6 months, at least 12 months, at least one year, or for more than one year.

Embodiment 36. The method according to any one of Embodiments 30-35, wherein the injectable composition is administered to the patient in combination with an effective amount of at least one additional therapeutic agent.

Embodiment 37. The method according to any one of Embodiments 30-36, wherein the injectable composition is administered to the patient in combination with an effective amount of at least one additional therapeutic agent chosen from redness reduction agents, NSAIDS, steroids, local anesthetics, antihistamines, anti-inflammatory agents, anti-allergens, or any combination thereof.

### Exemplary Container Closure Systems and Kits:

Embodiment 38. A container closure system containing an injectable composition comprising (a) at least one tetracycline antibiotic and (b) at least one semifluorinated alkane.

Embodiment 39. The container closure system of Embodiment 50, wherein the injectable composition is the injectable composition according to any of Embodiments 1-30.

Embodiment 40. The container closure system according to any one of claims 38-39, wherein the container closure system is chosen from vials, ampoules, bottles, tubes, syringes, and dispenser packages.

Embodiment 41. The container closure system according to any one of claims 38-40, wherein the container closure system is a polypropylene (PP) container closure system.

Embodiment 42. A kit comprising: (i) a formulation container closure system containing an injectable composition comprising (a) at least one tetracycline antibiotic and (b) at least one semifluorinated alkane and (ii) a formulation container closure system containing a delivery vehicle for dilution or suspension of the injectable composition in the formulation container.

Embodiment 43. The kit of Embodiment 42, wherein the injectable composition is the injectable composition according to any of Embodiments 1-30.

Embodiment 44. The kit according to any one of claims 42-43, wherein the formulation container closure system is chosen from vials, ampoules, and syringes.

Embodiment 45. The kit according to any one of claims 42-44, wherein the formulation container closure system is a PP pre-filled syringe.

### EXAMPLES

The following examples are intended to be illustrative and are not meant in any way to limit the scope of the disclosure.

### Example 1

A first composition was a nonaqueous suspension in accordance with the present application, comprising both at least one tetracycline antibiotic (doxycycline) and at least one semifluorinated alkane (F6H8), the contents of which are shown in Table 1 below:

TABLE 1

| Ingredient | | | |
|---|---|---|---|
| Inventive Composition | CAS | UNII | % w/w |
| Doxycycline monohydrate | 17086-28-1 | N12000U13O | 1.12 |
| perfluorohexyloctane (F6H8) | 133331-77-8 | 7VYX4ELWQM | 98.96 |

1. An injectable composition comprising: (a) at least one tetracycline antibiotic and (b) at least one semifluorinated alkane.

2. The injectable composition according to any one of claim 1, wherein the at least one tetracycline antibiotic is chosen from doxycycline, demeclocycline, minocycline, tetracycline, or any combination thereof.

3. The injectable composition according to claim 1, wherein the at least one tetracycline antibiotic comprises doxycycline.

4. The injectable composition according to claim 1, wherein the at least one tetracycline antibiotic consists essentially of doxycycline.

5. The injectable composition according to claim 4, wherein the at least one tetracycline antibiotic is included in the composition at a concentration of from about 0.005% (w/w) to about 5% (w/w).

6. The injectable composition according to claim 1, wherein the at least one semifluorinated alkane is a compound composed of at least one perfluorinated hydrocarbon segment (F-segment) and at least one non-fluorinated hydrocarbon segment (H-segment) according to the formula FnHm, wherein the F-segments and the H-segments are linear or branched and wherein n and m, which may be the same or different, are independently chosen from 3 to 20 carbon atoms.

7. The injectable composition according to claim 1, wherein the at least one semifluorinated alkane is chosen from F4H5, F4H6, F5H6, F5H7, F6H6, F6H7, F6H8, or any combination thereof.

8. The injectable composition according to claim 1, wherein the at least one semifluorinated alkane comprises 1-perfluorohexyl-octane (F6H8).

9. The injectable composition according to claim 1, wherein the at least one semifluorinated alkane consists essentially of 1-perfluorohexyl-octane (F6H8).

10. The injectable composition according to claim 1, wherein (a) the at least one tetracycline antibiotic is chosen from doxycycline, demeclocycline, minocycline, tetracycline, or any combination thereof and (b) the at least one semifluorinated alkane is chosen from F4H5, F4H6, F5H6, F5H7, F6H6, F6H7, F6H8, or any combination thereof.

11. The injectable composition according to claim 1, wherein (a) the at least one tetracycline antibiotic comprises doxycycline and (b) the at least one semifluorinated alkane comprises F6H8.

12. The injectable composition according to claim 1, wherein (a) the at least one tetracycline antibiotic consists essentially of doxycycline and (b) the at least one semifluorinated alkane consists essentially of F6H8.

13. The injectable composition according to claim 1, wherein the composition is liquid.

14. The injectable composition according to claim 13, wherein the composition is nonaqueous.

15. The injectable composition according to claim 14, wherein the composition is stable.

16. The injectable composition according to claim 1, wherein the composition further comprises at least one non-therapeutic component and/or pharmaceutically acceptable excipient.

17. The injectable composition according to claim 1, wherein the composition further comprises at least one additional therapeutic agent chosen from redness reduction agents, NSAIDS, steroids, local anesthetics, antihistamines, anti-inflammatory agents, anti-allergens, or any combination thereof.

18. A method for the treatment or temporary prevention of malar edema comprising administering via injection to a patient in need thereof an effective amount of an injectable composition comprising (a) at least one tetracycline antibiotic and (b) at least one semifluorinated alkane.

19. The method according to claim 18, wherein (a) the at least one tetracycline antibiotic is chosen from doxycycline, demeclocycline, minocycline, tetracycline, or any combination thereof and (b) the at least one semifluorinated alkane is chosen from F4H5, F4H6, F5H6, F5H7, F6H6, F6H7, F6H8, or any combination thereof.

20. The method according to claim 19, wherein (a) the at least one tetracycline antibiotic comprises doxycycline and (b) the at least one semifluorinated alkane comprises F6H8.

21. The method according to claim 20, wherein (a) the at least one tetracycline antibiotic consists essentially of doxycycline and (b) the at least one semifluorinated alkane consists essentially of F6H8.

22. The method according to claim 1, wherein the injectable composition is nonaqueous.

23. The method according to claim 1, wherein the injectable composition is administered to the patient in combination with an effective amount of at least one additional therapeutic agent chosen from redness reduction agents, NSAIDS, steroids, local anesthetics, antihistamines, anti-inflammatory agents, anti-allergens, or any combination thereof.
